(19) United States
(12) Patent Application Publication
(10) Pub. No.: US 2020/0352665 A1
(43) Pub. Date: Nov. 12, 2020

(54) INFORMATION PROCESSING DEVICE, INFORMATION PROCESSING METHOD, AND PROGRAM

(71) Applicant: SONY CORPORATION, TOKYO (JP)

(72) Inventors: YUKI ITOTANI, TOKYO (JP); RYUTA HORIE, SAITAMA (JP); TAKASHI KITO, KANAGAWA (JP); KAZUO HONGO, CHIBA (JP)

(21) Appl. No.: 16/962,332

(22) PCT Filed: Dec. 27, 2018
(86) PCT No.: PCT/JP2018/047999

§ 371 Date: Jul. 15, 2020

(30) Foreign Application Priority Data
Jan. 30, 2018 (JP) 2018-013616

(51) Int. Cl. A61B 34/37; A61B 34/00; A61B 34/35; B25J 13/08

(52) U.S. Cl. CPC A61B 34/37; B25J 13/085; A61B 34/35; A61B 34/76

(57) ABSTRACT

[Overview] [Problem to be Solved] To provide an information processing device that makes it possible to make a user feel less strange. [Solution] An information processing device including: a first acquisition unit that acquires a vibration signal measured by a vibration sensor included in a slave apparatus; a second acquisition unit that acquires a measurement result regarding distance between a target of sensing of the vibration sensor and a contact part of the slave apparatus; and a controller that outputs an output signal to a master apparatus. The contact part comes into contact with the target. The output signal is obtained by applying a weight to the vibration signal. The weight corresponds to the measurement result.

### TECHNICAL FIELD

The present disclosure relates to an information processing device, an information processing method, and a program.

### BACKGROUND ART

In recent years, as surgical operation systems used when endoscopic surgery is carried out, master-slave systems have been known that make it possible to approach affected sites without making large incisions on the bodies of patients. In such a system, a surgeon (user) such as a doctor operates a master apparatus including an input interface, and a slave apparatus including a medical instrument such as forceps or tweezers is remotely operated in accordance with the operation of the surgeon. The slave apparatus is configured, for example, as an arm apparatus with a surgical instrument held at the front end, and allows the surgical instrument to change the position or attitude in the abdomen.

In a case where a tactile sensation caused by a surgical instrument coming into contact with a patient is not transmitted to a surgeon in such a system, the surgeon may damage biological tissue of the patient without noticing that the surgical instrument is in contact with the patient. It is therefore desirable that a tactile sensation caused by a surgical instrument coming into contact with a patient be transmitted to a surgeon. Examples of techniques for transmitting a tactile sensation caused by a surgical instrument coming into contact with a patient to a surgeon include a technique for transmitting a tactile sensation to a surgeon as vibration or the like by providing a slave apparatus with a sensor that measures a tactile sensation and transmitting information regarding the tactile sensation measured by the sensor to a master apparatus side. The above-described technique, however, transmits, in addition to vibration regarding a tactile sensation, vibration (that is also referred to as vibration noise below) unrelated to the contact to the surgeon as well. The vibration noise includes the vibration of a motor of a slave apparatus, vibration in the installation place, vibration caused by noise, and the like. In connection with this, Patent Literature 1 below discloses a technique for reducing, by filtering, vibration noise included in a tactile sensation transmitted to a surgeon.

### CITATION LIST

### Patent Literature

PTL 1: Japanese Unexamined Patent Application Publication No. 2016-214715

### SUMMARY OF THE INVENTION

### Problems to be Solved by the Invention

The above-described technique in Patent Literature 1 is, however, configured to transmit vibration measured by a sensor to a master apparatus regardless of the positional relationship between a surgical instrument and a patient (e.g., biological tissue inside the body cavity and the skull). As a result, the master apparatus presents vibration based on similar vibration noise to a surgeon, for example, both when the surgical instrument and the patient are in contact with each other and when the surgical instrument and the patient are not in contact with each other. This may make the surgeon feel strange.

The present disclosure proposes a novel and improved information processing device, information processing method, and program each of which makes it possible to make a user feel less strange.

### Means for Solving the Problems

According to the present disclosure, there is provided an information processing device including: a first acquisition unit that acquires a vibration signal measured by a vibration sensor included in a slave apparatus; a second acquisition unit that acquires a measurement result regarding distance between a target of sensing of the vibration sensor and a contact part of the slave apparatus; and a controller that outputs an output signal to a master apparatus. The contact part comes into contact with the target. The output signal is obtained by applying a weight to the vibration signal. The weight corresponds to the measurement result.

In addition, according to the present disclosure, there is provided an information processing method that is executed by a processor. The information processing method includes: acquiring a vibration signal measured by a vibration sensor included in a slave apparatus; acquiring a measurement result regarding distance between a target of sensing of the vibration sensor and a contact part of the slave apparatus; and outputting an output signal to a master apparatus. The contact part coming into contact with the target. The output signal is obtained by applying a weight to the vibration signal. The weight corresponds to the measurement result.

In addition, according to the present disclosure, a program for causing a computer to function as a first acquisition unit that acquires a vibration signal measured by a vibration sensor included in a slave apparatus, a second acquisition unit that acquires a measurement result regarding distance between a target of sensing of the vibration sensor and a contact part of the slave apparatus, and a controller that outputs an output signal to a master apparatus. The contact part comes into contact with the target. The output signal is obtained by applying a weight to the vibration signal. The weight corresponds to the measurement result.

### Effects of the Invention

As described above, according to the present disclosure, it is possible to make a user feel less strange.

It is to be noted that the above-described effects are not necessarily limitative. Any of the effects indicated in this description or other effects that may be understood from this description may be exerted in addition to the above-described effects or in place of the above-described effects.

### BRIEF DESCRIPTION OF DRAWING

FIG. 1 is an explanatory diagram illustrating an overview of an information processing system according to an embodiment of the present disclosure.

FIG. 2 is a block diagram illustrating an internal configuration example of a slave apparatus according to the embodiment of the present disclosure.

FIG. 3 is a block diagram illustrating a configuration example of an output control unit according to a first embodiment of the present disclosure.

FIG. 4 is an explanatory diagram illustrating a temporal weight change according to the first embodiment of the present disclosure.

FIG. 5 is an explanatory diagram illustrating waveforms of an input signal and an output signal according to the first embodiment of the present disclosure.

FIG. 6 is a flowchart illustrating an operation example of an information processing device according to the first embodiment of the present disclosure.

FIG. 7 is a block diagram illustrating a configuration example of an output control unit according to a second embodiment of the present disclosure.

FIG. 8 is an explanatory diagram illustrating a temporal distance change and a temporal weight change according to the second embodiment of the present disclosure.

FIG. 9 is an explanatory diagram illustrating waveforms of an input signal and an output signal according to the second embodiment of the present disclosure.

FIG. 10 is an explanatory diagram illustrating a flowchart illustrating an operation example of an information processing device according to the second embodiment of the present disclosure.

FIG. 11 is a block diagram illustrating a configuration example of an output control unit according to a third embodiment of the present disclosure.

FIG. 12 is a block diagram illustrating a hardware configuration example of the slave apparatus according to the embodiment of the present disclosure

### MODES FOR CARRYING OUT THE INVENTION

The following describes a preferred embodiment of the present disclosure in detail with reference to the accompanying drawings. It is to be noted that, in this description and the accompanying drawings, components that have substantially the same functional configuration are indicated by the same reference signs, and thus redundant description thereof is omitted.

It is to be noted that description is given in the following order.

### 1. Overview of Information Processing System

2. Slave Apparatus according to the Present Embodiment

### 3. First Embodiment

### 4. Second Embodiment

### 5. Third Embodiment

### 6. Modification Examples

### 7. Hardware Configuration

### 8. Conclusion

### 1. OVERVIEW OF INFORMATION PROCESSING SYSTEM

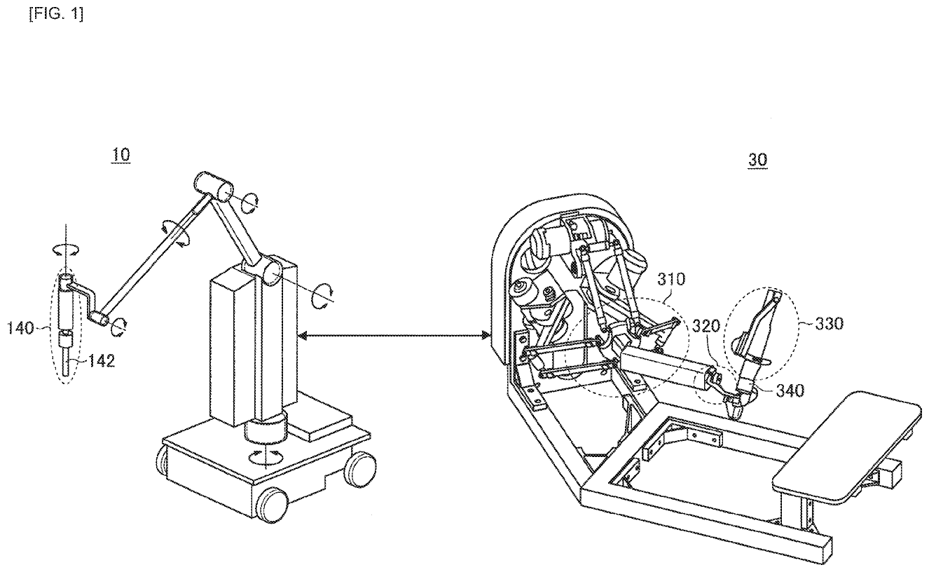

The following describes an overview of an information processing system according to an embodiment of the present disclosure with reference to FIG. 1. It is to be noted that a master-slave medical robot system is used as an example to describe an overview of the information processing system according to the embodiment of the present disclosure.

FIG. 1 is an explanatory diagram illustrating the overview of the information processing system according to the embodiment of the present disclosure. As illustrated in FIG. 1, the information processing system includes a slave apparatus 10 and a master apparatus 30. The slave apparatus 10 is an apparatus including a medical instrument such as forceps or tweezers that are remotely operated in accordance with an operation of a surgeon (who is also referred to as user below) on the master apparatus 30. In addition, the master apparatus 30 is an apparatus including an input interface that is operated by a surgeon such as a doctor.

For the information processing system, bilateral control is adopted as an example. The bilateral control is feedback control for matching the input interface, the position of the surgical instrument, and the force states to each other between the master apparatus and the slave apparatus. For example, when a surgeon operates the input interface, the position of the surgical instrument is moved in accordance with the operation. When the position of the surgical instrument is moved and the surgical instrument comes into contact with a patient, the force of the contact is fed back to the input interface.

It is to be noted that the slave apparatus 10 and the master apparatus 30 are coupled in any communication scheme. For example, the slave apparatus 10 and the master apparatus 30 are coupled through wired communication or wireless communication. In addition, for example, the slave apparatus 10 and the master apparatus 30 may be configured to directly establish communication, or establish communication via a network (or another apparatus).

### (1) Slave Apparatus 10

The slave apparatus 10 is a force sensation presenting apparatus that presents the force and vibration of the contact between an affected site (that is also referred to as target below) of a patient in surgery and a part (that is also referred to as contact part below) of the slave apparatus 10 that comes into contact with the target to the master apparatus 30. It is to be noted that an information processing device according to the embodiment of the present disclosure is applied to the slave apparatus 10.

The slave apparatus 10 is an apparatus (apparatus having a link mechanism including an active joint) for moving in association with a motion of the master apparatus 30, for example. The slave apparatus 10 includes one or more active joints and a link coupled to the active joints. The slave apparatus 10 includes, for example, motion sensors for measuring motions of the active joints at the respective positions corresponding to the active joints. Examples of the above-described motion sensors include an encoder and the like.

In addition, the slave apparatus 10 includes, for example, driving mechanisms for driving the active joints at the respective positions corresponding to the active joints. Examples of the above-described driving mechanisms include a motor and a driver.

A front end part 140 is the front end portion of the arm of the slave apparatus 10 illustrated in FIG. 1. The front end part 140 includes a contact part 142 at which a surgical instrument comes into contact with a patient. The front end part 140 is provided with various sensors. Examples of the various sensors include an origin sensor, a Limit sensor, an encoder, a force sensor, a vibration sensor, a distance measuring sensor, and the like. For example, the front end part 140 includes a force sensor. The force sensor measures force (that is also referred to as front end force below) applied to the contact part 142 when the contact part 142 comes into contact with a patient.

However, in a case of the slave apparatus according to the present embodiment, the force sensor also measures, in addition to the front end force, the gravity of the arm and the inertia force generated along with the movement of the arm. The force measured by the force sensor thus includes the front end force, the gravity, and the inertia force. It is to be noted that the following also refers to the force including the front end force, gravity, and inertia force measured by the force sensor as external force.

It is to be noted that the places where each of the above-described various sensors are installed at the front end part 140 are not limited in particular, but the various sensors may be installed in any places at the front end part 140. For example, the vibration sensor and the distance measuring sensor may be installed at the contact part 142 of the front end part 140. Alternatively, the vibration sensor and the distance measuring sensor may be installed in places other than the contact part 142 of the front end part 140.

### (2) Master Apparatus 30

The master apparatus 30 is a force sensation presenting apparatus that has functions of performing drive control on the slave apparatus 10, and presenting a vibration signal measured by a sensor of the slave apparatus 10 to a user. The master apparatus 30 is an apparatus (apparatus having a link mechanism including a passive joint) including one or more active joints including passive joints and a link coupled to the joints, for example. The master apparatus 30 includes, for example, an operation body 330 and a force sensor 340. The operation body 330 is provided to a link coupled to a passive joint. The force sensor 340 measures force applied to the operation body 330. In addition, the operation body 330 includes a vibration source for transmitting vibration fed back from the slave apparatus to a surgeon as a tactile sensation.

Here, examples of the force sensor 340 according to the present embodiment include any sensor that is able to measure force applied to the operation body 330, such as a “force sensation sensor having any system such as a system in which a strain gauge is used” or a “tactile sensor having any system such as a system in which a tactile sensation is obtained by using a piezoelectric element, a microphone, and the like to measure vibration. The master apparatus 30 is driven with electric power supplied from an internal power supply (not illustrated) such as a battery or electric power supplied from an external power supply of the master apparatus 30.

In addition, the master apparatus 30 includes, for example, motion sensors for measuring motions of the joints at the respective positions corresponding to the joints. Examples of the above-described motion sensors include an encoder and the like.

Further, the master apparatus 30 includes, for example, driving mechanisms for driving the active joints at the respective positions corresponding to the joints. Examples of the above-described driving mechanisms include a motor and a driver.

The master apparatus 30 illustrated in FIG. 1 is illustrated as an example of an apparatus whose three translation axes are achieved by an active joint part 310 that is driven by a motor, and whose three attitude axes are achieved by a passive joint part 320. In addition, the operation body 330 is provided to a link coupled to the passive joint part 320. In addition, the force sensor 340 that measures force applied to the operation body 330 is provided to the base portion of the operation body 330.

Here, FIG. 1 illustrates an example in which the operation body 330 provided to the master apparatus 30 is a stylus-shaped operation device, but the operation body 330 according to the present embodiment is not limited to the example illustrated in FIG. 1. Examples of the operation body 330 according to the present embodiment include an operation device having any shape such as a glove-shaped operation device. In addition, the operation body 330 according to the present embodiment may be any operation device that is applicable to a haptic device. In addition, the master apparatus 30 may have a structure in which the operation body 330 is replaceable. It is to be noted that the components of the master apparatus 30 according to the present embodiment are not limited to the example illustrated in FIG. 1, but the master apparatus 30 may have any components.

### (3) Reduction in Vibration Noise

As described above, vibration measured by the vibration sensor provided to the slave apparatus 10 is outputted from the vibration source provided to the master apparatus 30. For example, in a case where the contact part 142 (i.e., medical instrument) of the slave apparatus 10 comes into contact with a target (i.e., patient), force and vibration based on the contact are fed back to a user who operates the master apparatus 30. This allows the user to sense the contact part 142 coming into contact with the target, and the user is thus prompted to more carefully operate the operation body 330. As a result, it is possible to reduce the risk of damage to the target.

However, the vibration sensor provided to the slave apparatus 10 also measures vibration (i.e., vibration noise) that is unrelated to the contact, such as the vibration of a motor provided to the slave apparatus 10, vibration in the installation place of the slave apparatus 10, and vibration caused by noise around the slave apparatus 10. The vibration noise occurs regardless of whether or not the contact part 142 of the slave apparatus 10 is in contact with a target. Therefore, even if the contact part 142 of the slave apparatus 10 is not in contact with a target, vibration noise is measured and fed back to a user, making the user feel strange.

Moreover, constant feedback of vibration noise makes it difficult for a user to sense the contact part 142 of the slave apparatus 10 coming into contact with a target. This may increase the risk of damage to a target.

Accordingly, the information processing system according to the present embodiment applies a weight corresponding to the distance between the contact part 142 of the slave apparatus 10 and a target to a vibration signal measured by the vibration sensor provided to the slave apparatus 10, and causes the master apparatus 30 to output the vibration signal. This causes the vibration noise weighted in accordance with the distance between the contact part 142 of the slave apparatus 10 and the target to be fed back to a user while the contact part 142 of the slave apparatus 10 and the target are not in contact with each other. It is therefore possible to reduce the above-described strangeness regarding vibration noise, and reduce the risk of damage to the target.

The overview of the information processing system according to the embodiment of the present disclosure has been described above with reference to FIG. 1. Subsequently, the slave apparatus according to the present embodiment is described.

### 2. SLAVE APPARATUS ACCORDING TO THE PRESENT EMBODIMENT

The following describes, in more detail, the slave apparatus 10 to which the information processing device according to the embodiment of the present disclosure is applied.

### <2.1. Internal Configuration Example of Slave Apparatus>

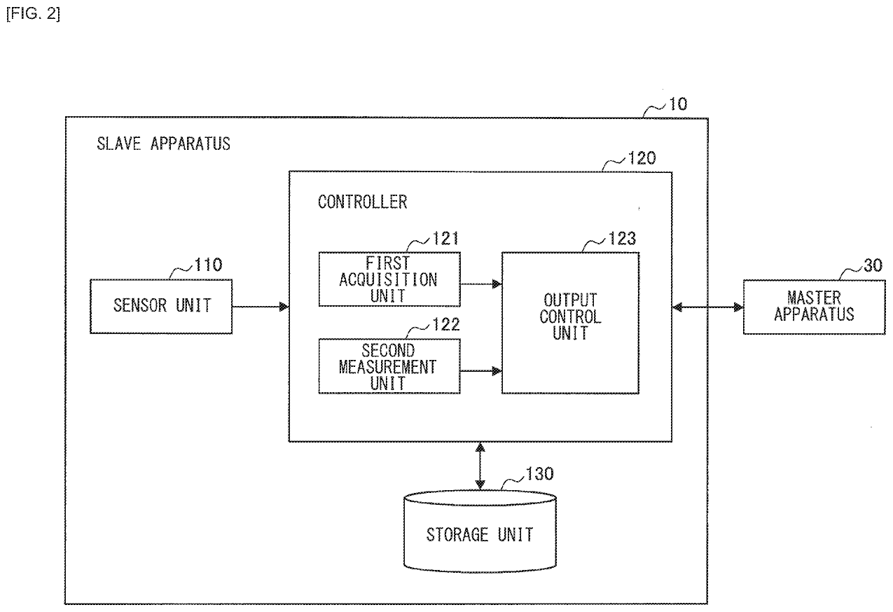

The following describes an internal configuration example of the slave apparatus according to the embodiment of the present disclosure with reference to FIG. 2. FIG. 2 is a block diagram illustrating the internal configuration example of the slave apparatus according to the embodiment of the present disclosure. As illustrated in FIG. 2, the slave apparatus 10 includes a sensor unit 110, a controller 120, and a storage unit 130. It is to be noted that the controller 120 has a function of the information processing device.

### (1) Sensor Unit 110

The sensor unit 110 includes a sensor for sensing the area around the slave apparatus 10. For example, the sensor unit 110 includes a vibration sensor for measuring vibration. Examples of the vibration sensor include an acceleration sensor, a microphone, and the like. In addition, the sensor unit 110 also includes a sensor for measuring information regarding the distance between a target and the contact part 142. Examples of the sensor for measuring the information regarding the distance include a force sensor, a contact sensor, a proximity sensor, a distance sensor, a biological sensor biological sensor (e.g., temperature sensor), and the like.

The sensor unit 110 uses the above-described sensor to measure information regarding distance, and transmits the measured information to a first acquisition unit 121 or a second acquisition unit 122 as a measurement result regarding distance.

It is to be noted that the number of sensors included in the sensor unit 110 is not limited, but any number of sensors may be included. In addition, the type of sensor included in the sensor unit 110 is not limited, but any type of sensor may be included.

### (2) Controller 120

The controller 120 has a function of controlling the operation of the slave apparatus 10. For example, the controller 120 controls a process of acquiring the information measured by the sensor unit 110. Specifically, the controller 120 distinguishes and acquires a vibration signal and information regarding distance from the information measured by the sensor unit 110.

In addition, the controller 120 has a function of controlling a process of outputting the acquired vibration signal. For example, the controller 120 decides a weight on the basis of the information regarding distance, and applies the weight to the vibration signal, thereby controlling the magnitude of the amplitude of the vibration signal for output.

To achieve the above-described function, the controller 120 according to the embodiment of the present disclosure includes the first acquisition unit 121, the second acquisition unit 122, and an output control unit 123 as illustrated in FIG. 2.

### (First Acquisition Unit 121)

The first acquisition unit 121 has a function of acquiring a vibration signal. For example, the first acquisition unit 121 acquires a vibration signal measured by the vibration sensor included in the sensor unit 110. More specifically, for example, in a case where the sensor unit 110 includes an acceleration sensor, the first acquisition unit 121 acquires a vibration signal on the basis of acceleration measured by the acceleration sensor. In addition, for example, in a case where the sensor unit 110 includes a microphone, the first acquisition unit 121 acquires a vibration signal on the basis of sound measured by the microphone. The first acquisition unit 121 then transmits the vibration signal to the output control unit 123 as an input signal.

### (Second Acquisition Unit 122)

The second acquisition unit 122 has a function of acquiring information regarding distance. For example, the second acquisition unit 122 uses a sensor included in the sensor unit 110 to acquire a measurement result regarding the distance between a target and the contact part 142.

More specifically, the second acquisition unit 122 acquires force (front end force) applied to the contact part 142 of the slave apparatus 10 and measured by the force sensor included in the sensor unit 110. However, the force acquired by the second acquisition unit 122 then acquires not only the front end force, but also inertia force. That is, the second acquisition unit 122 acquires external force. The second acquisition unit 122 then acquires the external force as a measurement result regarding distance. The second acquisition unit 122 then transmits the measurement result to the output control unit 123 as an input signal.

It is to be noted that the above-described measurement result acquired by the second acquisition unit 122 is not information directly indicating the distance between the target and the contact part 142. The information is, however, used to estimate the distance between the target and the contact part 142. For example, the output control unit 123 uses the information in a determination of contact described below to determine whether or not the target and the contact part 142 are in contact with each other. In a case where it is determined that the target and the contact part 142 are in contact with each other, the distance between the target and the contact part 142 is estimated to be 0. In addition, in a case where it is determined that the target and the contact part 142 are not in contact with each other, the distance between the target and the contact part 142 is estimated not to be 0. Even if the information is not information directly indicating the distance between the target and the contact part 142, the output control unit 123 is thus able to estimate the distance.

Even if the information measured by the sensor unit 110 is not information directly indicating the distance, the second acquisition unit 122 may therefore acquire the information measured by the sensor unit 110 as a measurement result regarding the distance as long as the information measured by the sensor unit 110 is usable as information for estimating the distance.

In addition, the second acquisition unit 122 may acquire the distance between a target and the contact part 142 measured by a sensor (e.g., distance sensor) included in the sensor unit 110 as a measurement result regarding the distance.

### (Output Control Unit 123)

The output control unit 123 has a function of controlling a process of outputting a vibration signal measured by the sensor unit 110 to the master apparatus 30. In controlling the output process of a vibration signal, the output control unit 123 makes, for example, a determination of the contact between a target and the contact part 142. More specifically, the output control unit 123 determines whether or not a target and the contact part 142 are in contact with each other, on the basis of a measurement result regarding the distance acquired by the second acquisition unit 122.

In addition, the output control unit 123 decides a weight on the basis of a result of the above-described determination of contact. For example, in a case where it is determined that the target and the contact part 142 are in contact with each other, the output control unit 123 decides a weight to output a vibration signal. In a case where it is determined that the target and the contact part 142 are not in contact with each other, the output control unit 123 decides a weight to cut off a vibration signal. It is to be noted that the output control unit 123 may decide a weight on the basis of a measurement result regarding distance acquired by the second acquisition unit 122 without making the above-described determination of contact in accordance with a component thereof.

In addition, the output control unit 123 applies the above-described weight to an input signal, and then outputs an output signal to the master apparatus 30. For example, the output control unit 123 outputs a signal obtained by multiplying an input signal by a weight as an output signal. When the output control unit 123 applies a weight to an input signal, the amplitude of an output signal has the magnitude corresponding to the magnitude of the weight.

It is noted that the above-described process of controlling the output of an output signal by the output control unit 123 may be performed in real time with respect to an input signal, or performed after an input signal is temporarily stored.

It is to be noted that a component of the output control unit 123 for achieving the above-described function depends on each of a plurality of embodiments described below, and the details thereof are thus described in each embodiment.

### (3) Storage Unit 130

The storage unit 130 is a device for storing information regarding the slave apparatus 10. For example, the storage unit 130 stores data outputted in a process of the controller 120 and data of various applications and the like.

The internal configuration example of the slave apparatus according to the embodiment of the present disclosure has been described above with reference to FIG. 2. Subsequently, an information processing system according to a first embodiment of the present disclosure is described.

### 3. FIRST EMBODIMENT

In an information processing device according to the first embodiment, an output control unit 123-1 of the controller 120 decides a weight on the basis of a determination of contact, and outputs an output signal to which the weight is applied to the master apparatus 30.

### <3.1. Configuration Example of Output Control Unit 123-1>

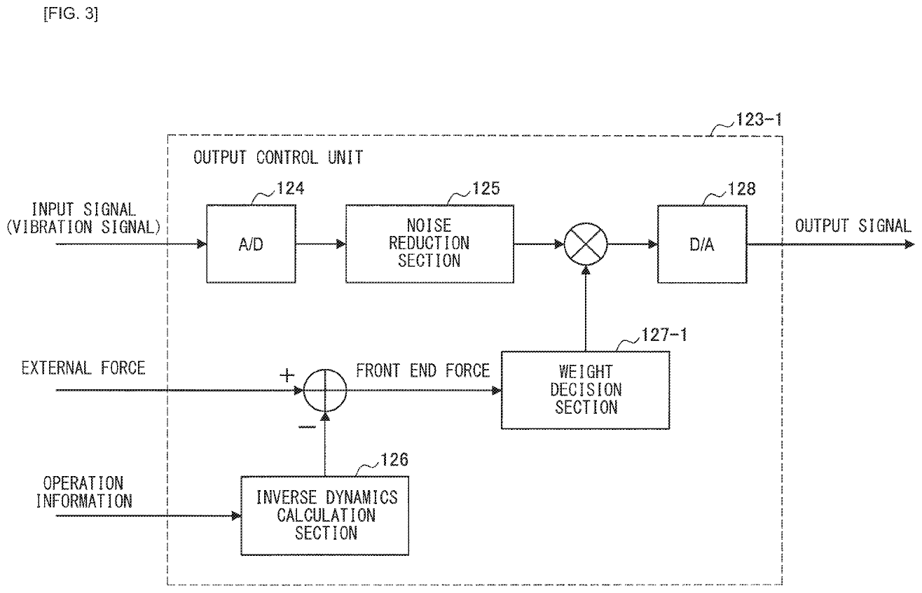

The following describes a configuration example of an output control unit according to the first embodiment of the present disclosure with reference to FIGS. 3 to 5. FIG. 3 is an explanatory diagram illustrating the configuration example of the output control unit according to the first embodiment of the present disclosure. As illustrated in FIG. 3, the output control unit 123-1 includes an A/D 124, a noise reduction section 125, an inverse dynamics calculation section 126, a weight decision section 127-1, and a D/A 128.

### (1) A/D 124

The A/D 124 is an analog-digital conversion circuit (A/D conversion circuit), and has a function of converting an analog signal into a digital signal. For example, the A/D 124 receives a vibration signal from the first acquisition unit 121 as an input signal, and converts the received vibration signal from an analog signal to a digital signal. The A/D 124 then outputs the converted digital signal to the noise reduction section 125.

### (2) Noise Reduction Section 125

The noise reduction section 125 has a function of removing a portion of vibration noise from an input signal. For example, the noise reduction section 125 removes, by using a filter, a frequency component corresponding vibration such as sound that a user does not sense as a tactile sensation, or a predetermined frequency component stored in advance from a vibration signal. More specifically, the noise reduction section 125 applies a filter to an input signal to remove noise in a predetermined frequency band. More specifically, for example, the noise reduction section 125 uses a low-pass filter (LPF: Low-Pass Filter) to cut off a vibration signal having a predetermined frequency or higher to remove noise from an input signal. The low-pass filter (LPF: Low-Pass Filter) cuts off a high-frequency signal and transmits only a low-frequency signal. The predetermined frequency here is an upper limit value of a frequency that a user is able to sense as a tactile sensation. For example, the predetermined frequency may be approximately 700 Hz. In addition, the predetermined frequency may be controlled, for example, in accordance with of the age, sex, and skin condition of a user, and in accordance with whether or not a user wears a glove. It is to be noted that the above-described predetermined frequency may be registered in advance in the storage unit 130.

In addition, for example, the noise reduction section 125 uses a high-pass filter (HPF: High-Pass Filter) to cut off a vibration signal having a predetermined frequency or lower to remove noise from an input signal. The high-pass filter (HPF: High-Pass Filter) cuts off a low-frequency signal and transmits only a high-frequency signal. The predetermined frequency here is a lower limit value of a frequency that a user is able to sense as a tactile sensation. For example, the predetermined frequency may be approximately 30 Hz. In addition, the predetermined frequency may be controlled, for example, in accordance with of the age, sex, and skin condition of a user, and in accordance with whether or not a user wears a glove.

In addition, the noise reduction section 125 removes, for example, the predetermined frequency component stored in advance from a vibration signal. More specifically, the storage unit 130 stores the frequency corresponding to the predetermined frequency component in advance. In a case where the frequency component corresponding to the frequency is inputted, the noise reduction section 125 removes the frequency component from the input signal. The noise reduction section 125 then outputs the input signal from which noise is removed to the D/A 128.

In this way, the noise reduction section 125 reduces noise to prevent the vibration source provided to the master apparatus 30 from outputting vibration in the frequency domain that does not correspond to a tactile sensation or vibration from a noise source whose frequency has been known.

It is to be noted that a filter used by the noise reduction section 125 is not limited to LPF or HPF, but may be any filter. In addition, a method for the noise reduction section 125 to reduce noise is not limited to a method that uses a filter, but may be any method.

### (3) Inverse Dynamics Calculation Section 126

The inverse dynamics calculation section 126 has a function of performing inverse dynamics calculation on operation information of the slave apparatus 10. Here, the operation information is a measurement result of the motion sensor included in the slave apparatus 10. For example, the inverse dynamics calculation section 126 acquires external force measured by the force sensor of the sensor unit 110 from the second acquisition unit 122, and corrects the external force with inverse dynamics calculation. The force sensor of the sensor unit 110 attempts to measure force (front end force) applied to the front end part 140. The force measured by the force sensor is, however, external force including gravity and inertia force in addition to the front end force. It is thus hard to consider that the force measured by the force sensor indicates accurate front end force. Accordingly, the use of a result of inverse dynamics calculation allows the inverse dynamics calculation section 126 to calculate more accurate front end force from the external force measured by the force sensor. This is because the inverse dynamics calculation allows the gravity and the inertia force to be obtained.

Here, inverse dynamics calculation is described. The inverse dynamics calculation section 126 performs inverse dynamics calculation on (θ, θ′, and θ″) that is a measurement result (i.e., operation information) of the motion sensor included in the slave apparatus 10. Here, (θ, θ′, and θ″) represents (angle of joint, angular velocity of joint, and angular acceleration of joint). In general, the dynamics of a robot like the slave apparatus 10 according to the present embodiment are expressed as the following Expression 1.

[Expression 1]

τ=J(τ)θ″+c(θ,θ′)+g(θ) (Expression 1)

Here, the left side of Expression 1 described above represents the torque value of each joint of the robot. In addition, the first term, the second term, and the third term on the right side of Expression 1 described above respectively represents an inertia term, a centrifugal force/Coriolis force term, and a gravity term.

The inverse dynamics calculation section 126 provides a virtual joint to the force sensor unit by a technique that uses inverse dynamics calculation, thereby calculating gravity/inertia force applied to the force sensor unit and subtracting the gravity/inertia force from the external force to calculate the front end force.

### (4) Weight Decision Section 127-1

The weight decision section 127-1 has a function of deciding a weight applied to a vibration signal. In the present embodiment, the weight decision section 127-1 performs a contact determination process, and performs a weight decision process based on a result of the contact determination process to decide a weight.

### (Contact Determination Process)

The weight decision section 127-1 determines whether or not the contact part 142 of the slave apparatus 10 and a target come into contact with each other. For example, the weight decision section 127-1 makes a determination of the contact between the contact part 142 of the slave apparatus 10 and a target on the basis of external force acquired by the second acquisition unit 122. More specifically, the weight decision section 127-1 makes a determination of the contact on the basis of front end force obtained by the inverse dynamics calculation section 126 correcting the external force with inverse dynamics calculation. The weight decision section 127-1 makes a determination of the contact, for example, on the basis of whether or not the front end force is greater than or equal to a predetermined threshold. If a result of the determination of the contact indicates that the front end force is greater than or equal to the predetermined threshold, the weight decision section 127-1 determines that the contact part 142 and the target part are in contact with each other. In addition, if the front end force is less than the predetermined threshold, the weight decision section 127-1 determines that the contact part 142 and the target part are not in contact with each other.

As described above, the use of front end force obtained by correcting external force with inverse dynamics calculation for a determination of contact allows the weight decision section 127-1 to obtain a result of a more accurate determination of contact as compared with a result of a determination of contact obtained by using external force as acquired from the second acquisition unit 122 for a determination of contact.

The following describes an example in which a measurement result of a sensor other than the force sensor is used for a determination of contact.

For example, in a case where the sensor unit 110 includes a contact sensor, the weight decision section 127-1 may determine being in contact in a case where the contact sensor measures contact with a target. In addition, the weight decision section 127-1 may determine being out of contact in a case where the contact sensor measures no contact with a target.

In addition, for example, in a case where the sensor unit 110 includes a proximity sensor, the weight decision section 127-1 may determine being in contact in a case where a value indicating the degree of proximity measured by the proximity sensor is greater than or equal to a predetermined threshold. In addition, the weight decision section 127-1 may determine being out of contact in a case where a value indicating the degree of proximity measured by the proximity sensor is less than the predetermined threshold.

In addition, for example, in a case where the sensor unit 110 includes a distance sensor, the weight decision section 127-1 may determine being in contact in a case where the distance measured by the distance sensor is less than a predetermined threshold. In addition, the weight decision section 127-1 may determine being out of contact in a case where the distance measured by the distance sensor is greater than or equal to the predetermined threshold.

In addition, for example, in a case where the sensor unit 110 includes a biological sensor, the weight decision section 127-1 may make a determination of the contact between the contact part 142 and a target on the basis of biological information of the target measured by the biological sensor. More specifically, for example, in a case where the sensor unit 110 uses a temperature sensor as a biological sensor, the weight decision section 127-1 may determine being in contact in a case where the temperature measured by the temperature sensor is higher than or equal to a predetermined threshold. In addition, the weight decision section 127-1 may determine being out of contact in a case where the temperature measured by the temperature sensor is lower than the predetermined threshold.

### (Weight Decision Process)

After a determination of contact, the weight decision section 127-1 decides a weight on the basis of a result of the determination of contact. The weight decision section 127-1 decides a large weight in a case where the contact part 142 and a target are in contact with each other. The weight decision section 127-1 decides a small weight in a case where the contact part 142 and a target are not in contact with each other. Specifically, in a case where it is determined as the determination of contact that the contact part 142 and a target are in contact with each other, a weight is decided to output an output signal. For example, the weight decision section 127-1 decides a weight as 1. In addition, in a case where it is determined as the determination of contact that the contact part 142 and a target are not in contact with each other, the weight decision section 127-1 decides a weight to cut off an output signal. For example, the weight decision section 127-1 decides a weight as 0. It is to be noted that a weight of 1 at the time when the contact part 142 and a target are in contact with each other is not limitative, but any value other than 0 may be decided as the weight.

As described above, the weight decision section 127-1 decides a weight on the basis of a result of a determination of contact to cause the output control unit 123-1 to output an output signal in a case where the contact part 142 is in contact with a target. In addition, the output control unit 123-1 outputs no output signal in a case where the contact part 142 is not in contact with a target. As a result, the master apparatus 30 presents vibration to a user in a case where the contact part 142 of the slave apparatus 10 and a target are in contact with each other. In contrast, the master apparatus 30 presents no vibration to a user in a case where the contact part 142 of the slave apparatus 10 and a target are not in contact with each other. It is therefore possible to make a user feel less strange, and reduce the risk of damage to the target.

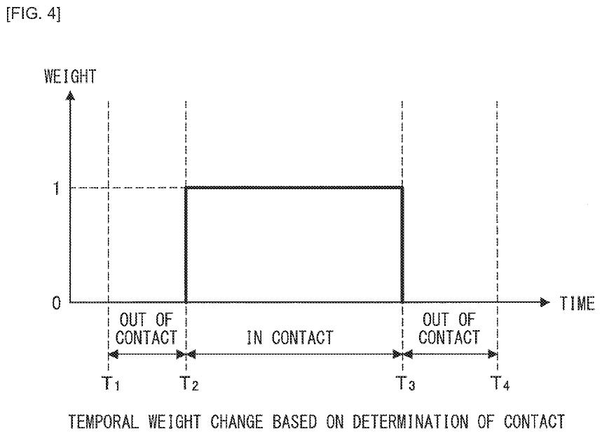

Here, the weight decision process according to the first embodiment is specifically described with reference to FIG. 4. FIG. 4 is an explanatory diagram illustrating a temporal weight change according to the first embodiment. The vertical axis of the graph illustrated in FIG. 4 represents a weight, and the horizontal axis represents time.

As illustrated in FIG. 4, for example, it is assumed that a result of a determination of contact by the weight decision section 127-1 indicates being out of contact from time T₁ to time T₂, being in contact from the time T₂ to time T₃, and being out of contact from the time T₃ to time T₄. The weight decision section 127-1 decides weight=0 from the time T₁ to the time T₂, weight=1 from the time T₂ to the T₃, and weight=0 from the time T₃ to the T₄ in accordance with the above-described result of the determination.

### (Weight Application Process)

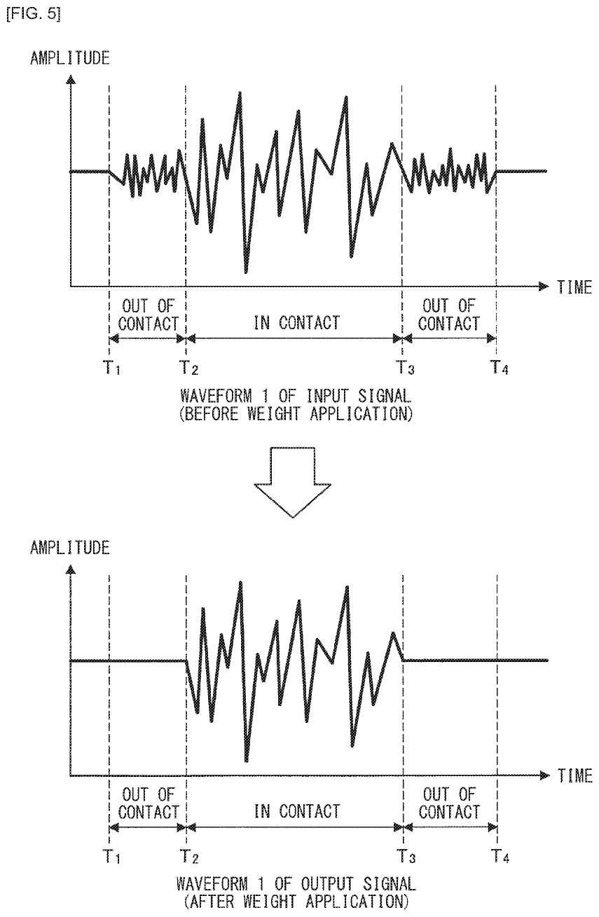

After a weight is decided, the weight decision section 127-1 applies the weight to a vibration signal (input signal). The following describes a specific example of a process of applying the weight decided in the above-described weight decision process to an input signal after a noise reduction process by the noise reduction section 125 with reference to FIG. 5. FIG. 5 is an explanatory diagram illustrating the waveform of an output signal according to the first embodiment of the present disclosure. FIG. 5 illustrates the respective graphs of a waveform 1 of an input signal acquired by the first acquisition unit 121, and the waveform 1 of an output signal in which a weight is applied to the input signal. The vertical axis of each graph illustrated in FIG. 5 represents amplitude, and the horizontal axis represents time.

As indicated by the waveform 1 of the input signal in FIG. 5, the input signal is measured from the time T₁ to the time T₂, from the time T₂ to the time T₃, and from the time T₃ to the time T₄. The input signal from the time T₁ to the time T₂ corresponds to vibration noise. The input signal from the time T₂ to the time T₃ corresponds to the vibration of a target and vibration noise. The input signal from the time T₃ to the time T₄ corresponds to vibration noise. The weight decision section 127-1 applies the weight decided in the above-described weight decision process to this input signal. For example, a weight is equal to 0 from the time T₁ to the time T₂. Thus, when the weight decision section 127-1 applies the weight to the input signal, the input signal is cut off and an output signal is outputted as 0. In addition, a weight is equal to 1 from the time T₂ to the time T₃. Thus, when the weight decision section 127-1 applies the weight to the input signal, the input signal is not cut off, but is outputted as it is as an output signal. A weight is equal to 0 from the time T₃ to the time T₄. Thus, when the weight decision section 127-1 applies the weight to the input signal, the input signal is cut off again and an output signal is outputted as 0.

It is to be noted that an input signal subjected to the weight application process is not limited to an input signal after the noise reduction process by the noise reduction section 125, but may be an input signal before the noise reduction process.

### (5) D/A 128

The D/A 128 is a digital-analog conversion circuit (D/A conversion circuit), and has a function of converting a digital signal into an analog signal. For example, the D/A 128 receives a digital signal transmitted from the noise reduction section 125. A weight is applied to the digital signal. The D/A 128 converts the digital signal to an analog signal. The D/A 128 then outputs the converted analog signal as an output signal.

It is to be noted that the timing of the conversion process by the D/A 128 is not limited to the timing indicated in the above-described example, but may be any timing. For example, the D/A 128 may perform a conversion process on a digital signal transmitted from the noise reduction section 125 before a weight is applied.

The configuration example of the output control unit 123-1 according to the first embodiment of the present disclosure has been described above with reference to FIGS. 3 to 5. Subsequently, an operation example of the slave apparatus 10 according to the first embodiment of the present disclosure is described.

### <3.2. Operation Example of Slave Apparatus 10>

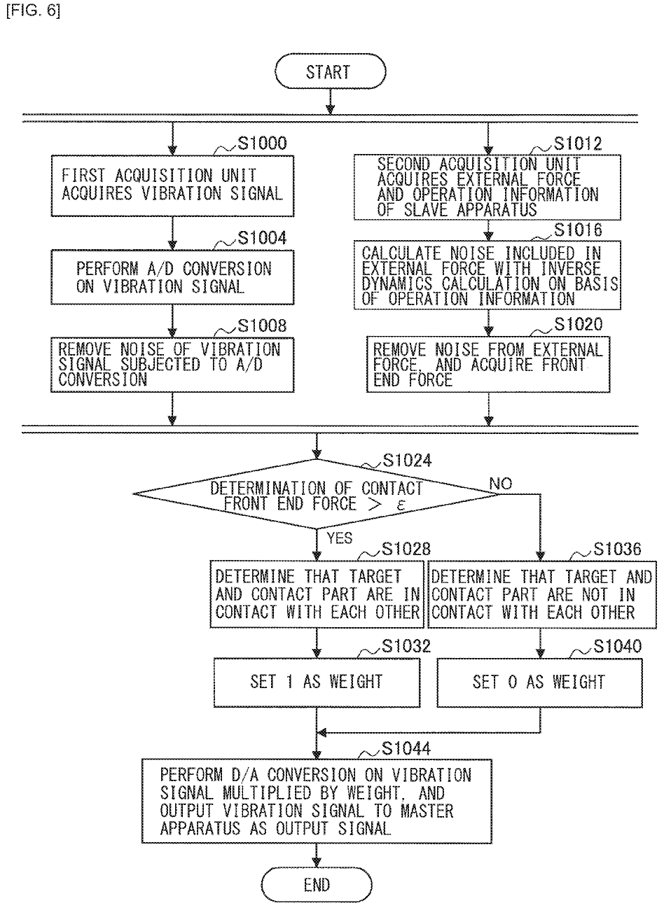

The following describes an operation example of the slave apparatus 10 according to the first embodiment of the present disclosure with reference to FIG. 6. FIG. 6 is a flowchart illustrating an operation example of the controller 120 of the slave apparatus 10 according to the first embodiment of the present disclosure.

First, the slave apparatus 10 comes into operation in accordance with an operation of a user on the master apparatus 30. The first acquisition unit 121 of the controller 120 then acquires a vibration signal measured by the sensor unit 110 (step S1000), and transmits the vibration signal to the output control unit 123-1. The A/D 124 of the output control unit 123-1 converts the vibration signal from an analog signal to a digital signal (step S1004), and transmits the converted vibration signal to the noise reduction section 125. The noise reduction section 125 removes, by filtering, noise from the vibration signal converted into a digital signal (step S1008).

In addition, in parallel with the above-described processes in steps S1000, 1004, and 1008, the second acquisition unit 122 of the controller 120 acquires external force measured by the sensor unit 110 and operation information of the slave apparatus 10 (step S1012). The second acquisition unit 122 then transmits the acquired external force and operation information to the output control unit 123-1. When the output control unit 123-1 receives the external force and the operation information, the inverse dynamics calculation section 126 of the output control unit 123-1 calculates the gravity and the inertia force included in the external force with inverse dynamics calculation on the basis of the operation information (step S1016). The output control unit 123-1 then removes the gravity and the inertia force from the external force, and acquires the front end force (step S1020).

After the parallel processes are finished, the weight decision section 127-1 confirms whether or not the front end force acquired in step S1020 satisfies a predetermined condition (front end force>threshold ε), and makes a determination of contact (step S1024). In a case where the front end force satisfies the predetermined condition (step S1024/YES), the weight decision section 127-1 determines that the contact part 142 of the slave apparatus 10 and the target are in contact with each other (step S1028). The weight decision section 127-1 then decides a weight as 1 (step S1032). In addition, in a case where the front end force does not satisfy the predetermined condition (step S1024/NO), the weight decision section 127-1 determines that the contact part 142 of the slave apparatus 10 and the target are not in contact with each other (step S1036). The weight decision section 127-1 then decides a weight as 0 (step S1040). After the weight is decided, the output control unit 123-1 outputs a vibration signal multiplied by the weight by the weight decision section 127-1 and converted from a digital signal to an analog signal by the D/A 128 to the master apparatus 30 as an output signal (step S1044).

The operation example of the slave apparatus 10 according to the first embodiment of the present disclosure has been described above with reference to FIG. 6.

The first embodiment of the present disclosure has been described above with reference to FIGS. 3 to 6. Subsequently, a second embodiment of the present disclosure is described.

### 4. SECOND EMBODIMENT

The information processing device according to the first embodiment decides a weight with a discrete value that depends on whether or not the contact part 142 and a target are in contact with each other, and applies the decided weight to a vibration signal. In contrast, the information processing device according to the second embodiment decides a weight with a continuous value corresponding to the distance itself between the contact part 142 and a target, and applies the decided weight to a vibration signal.

### <4.1. Configuration Example of Output Control Unit 123-2>

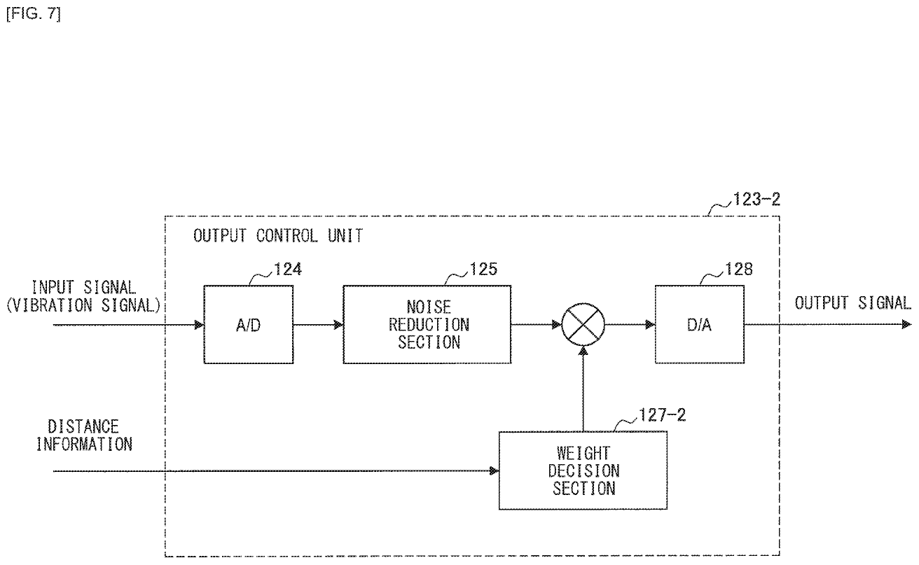

The following describes a configuration example of an output control unit according to the second embodiment of the present disclosure with reference to FIGS. 7 to 9. FIG. 7 is an explanatory diagram illustrating the configuration example of the output control unit according to the second embodiment of the present disclosure. As illustrated in FIG. 7, the output control unit 123-2 includes the A/D 124, the noise reduction section 125, a weight decision section 127-2, and the D/A 128. It is to be noted that the output control unit 123-2 does not make the determination of contact that is based on front end force in the second embodiment. This removes the inverse dynamics calculation section 126 from the components of the output control unit 123-2.

### (1) A/D 124

The function of the A/D 124 is the same as the function described in <3.1. Configuration Example of Output Control Unit 123-1>, and the description thereof is omitted in this chapter.

### (2) Noise Reduction Section 125

The function of the noise reduction section 125 is the same as the function described in <3.1. Configuration Example of Output Control Unit 123-1>, and the description thereof is omitted in this chapter.

### (3) Weight Decision Section 127-2

Different from the weight decision section 127-1 according to the first embodiment, the weight decision section 127-2 makes no determination of contact, but decides a weight on the basis of distance information. The distance information is information indicating the distance itself between the contact part 142 and a target.

### (Weight Decision Process)

The weight decision section 127-2 has a function of deciding a weight on the basis of distance information. For example, as a measurement result acquired by the second acquisition unit 122 regarding the distance between the contact part 142 of the slave apparatus 10 and a target, the weight decision section 127-2 acquires the distance between the contacted object and the target, and continuously changes the weight in accordance with the distance. More specifically, for example, the weight decision section 127-2 determines higher priority for outputting a vibration signal measured by the vibration sensor of the sensor unit 110 and decides a large weight because the target and the contact part 142 are positioned closer to each other with a decrease in the distance. In addition, for example, the weight decision section 127-2 determines lower priority for presenting a vibration signal measured by the vibration sensor of the sensor unit 110 and decides a small weight because the target and the contact part 142 are positioned farther from each other with an increase in the distance.

It is to be noted that the weight decision section 127-2 may make not only the determination based on the distance between the contact part 142 and a target, but also the determination based on a human sensing limit to vibration amplitude to decide a weight. For example, vibration whose vibration amplitude has magnitude far from a sensing limit value within the range of vibration amplitude that a human is able to sense is vibration that a user is highly likely to be able to sense. This causes the weight decision section 127-2 to determine high priority for presenting the vibration to a user, and decide a large weight. In addition, for example, vibration whose vibration amplitude has magnitude close to the sensing limit value is vibration that a user is less likely to be able to sense. This causes the weight decision section 127-2 to determine low priority for presenting the vibration to a user, and decide a small weight.

As described above, the weight decision section 127-2 decides a weight on the basis of the distance between the contact part 142 and a target to allow the output control unit 123-1 to output an output signal even in a case where the contact part 142 is not in contact with the target. This allows the slave apparatus 10 to present information at the time when the contact part 142 and a target are not in contact with each other to the master apparatus 30 as vibration. That is, the slave apparatus 10 is able to present sound or vibration generated around the target to the master apparatus 30 as vibration.

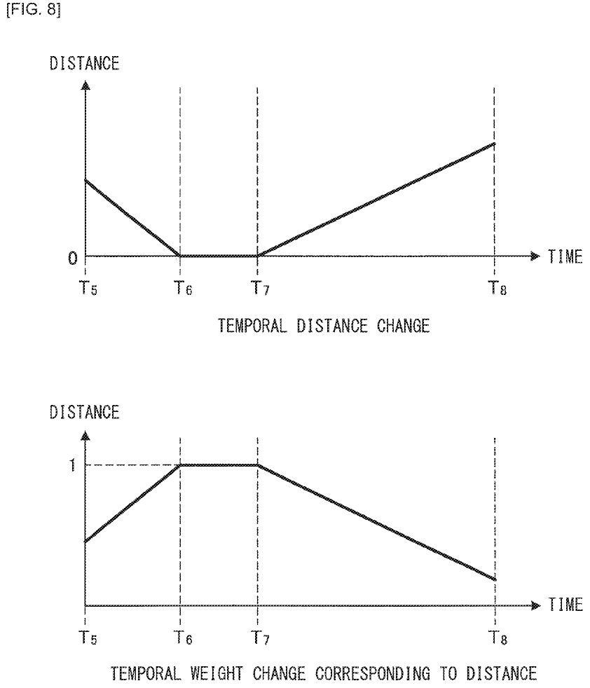

Here, the weight decision process according to the second embodiment is specifically described with reference to FIG. 8. FIG. 8 is an explanatory diagram illustrating a temporal distance change and a temporal weight change according to the second embodiment. The vertical axis of the graph illustrated in FIG. 8 that indicates a temporal distance change represents distance, and the horizontal axis represents time. In addition, the vertical axis of the graph indicating a temporal weight change corresponding to distance represents a weight, and the horizontal axis represents time.

It is assumed, for example, that the distance between a target and the contact part 142 acquired by the second acquisition unit decreases over time from time T₅ to time T₆, is 0 from the time T₆ to time T₇, and increases over time from the time T₇ to time T₈ as illustrated in FIG. 8. The weight decision section 127-2 decides a weight that increases over time from the time T₅ to the time T₆ in accordance with the above-described temporal distance change. In addition, the weight decision section 127-2 decides a weight as 1 because the distance is constantly 0, that is, the target and the contact part 142 are in contact with each other from the time T₆ to the time T₇. In addition, the weight decision section 127-2 decides a weight that decreases over time from the time T₇ to the time T₈ in accordance with the above-described temporal distance change.

### (Weight Application Process)

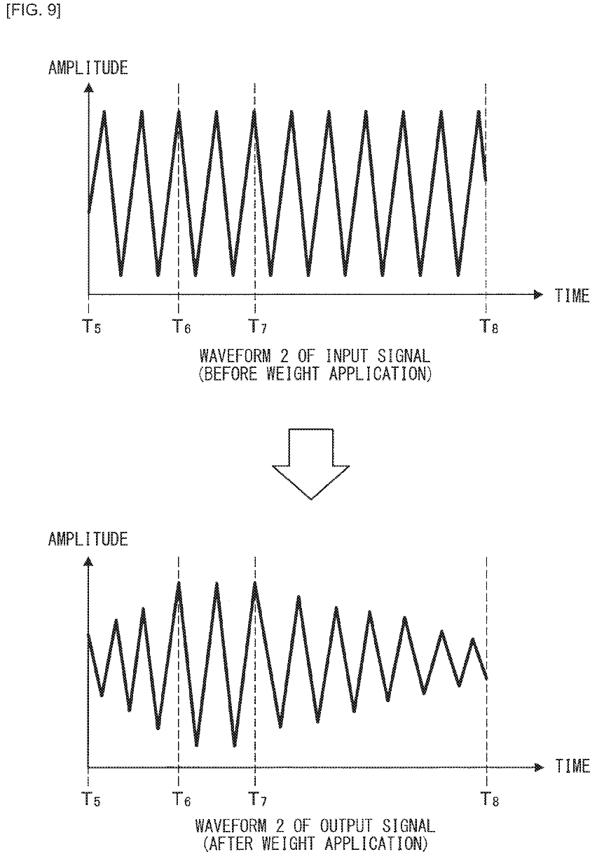

After a weight is decided, the weight decision section 127-2 applies the weight to a vibration signal (input signal). The following describes a specific example of a process of applying the weight decided in the above-described weight decision process to an input signal with reference to FIG. 9. FIG. 9 is an explanatory diagram illustrating the waveform of an output signal according to the second embodiment of the present disclosure. FIG. 9 illustrates the respective graphs of a waveform 2 of an input signal acquired by the first acquisition unit 121, and the waveform 2 of an output signal in which a weight is applied to the input signal. The vertical axis of each graph illustrated in FIG. 9 represents amplitude, and the horizontal axis represents time.

As indicated by the waveform 2 of the input signal in FIG. 9, the input signal having constant amplitude is measured from the time T₅ to the time T₆, from the time T₆ to the time T₇, and from the time T₇ to the time T₈. The input signal from the time T₅ to the time T₈ corresponds to vibration generated around a target. The input signal from the time T₅ to the time T₆ corresponds to vibration noise. The input signal from the time T₆ to the time T₇ corresponds to the vibration of a target and vibration noise. The input signal from the time T₇ to the time T₈ corresponds to vibration noise. The weight decision section 127-2 applies the weight decided in the above-described weight decision process to this input signal. For example, a weight increases over time from the time T₅ to the time T₆, and the amplitude of the waveform 2 of an output signal after the weight is applied thus increases over time. In addition, the weight is constantly 1 from the time T₆ to the time T₇, and the amplitude of the waveform 2 of an output signal after the weight is applied thus has the same magnitude as the magnitude of the amplitude of the waveform 2 of the input signal. In addition, a weight decreases over time from the time T₇ to the time T₈, and the amplitude of the waveform 2 of an output signal after the weight is applied thus decreases over time.

### (4) D/A 128

The function of the D/A 128 is the same as the function described in <3.1. Configuration Example of Output Control Unit 123-1>, and the description thereof is omitted in this chapter.

The configuration example of the output control unit 123-2 according to the second embodiment of the present disclosure has been described above with reference to FIGS. 7 to 9. Subsequently, an operation example of the slave apparatus 10 according to the second embodiment of the present disclosure is described.

### <4.2. Operation Example of Slave Apparatus 10>

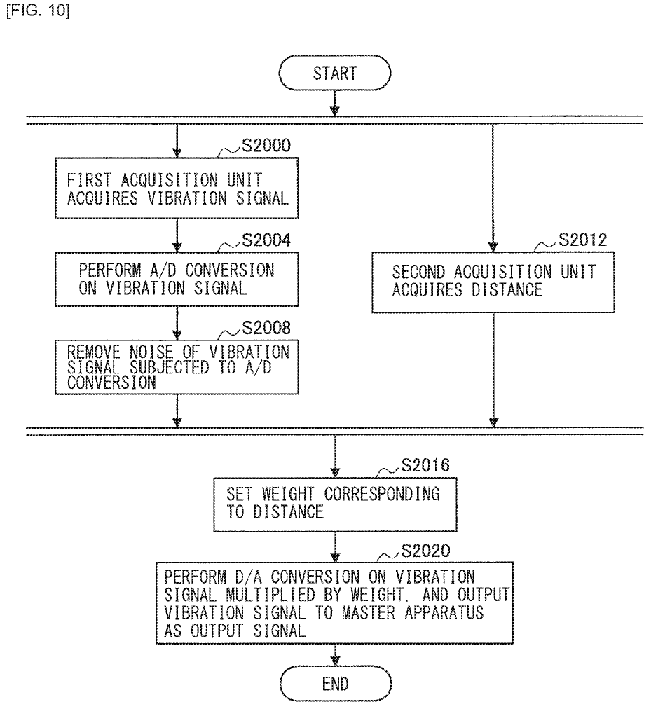

The following describes an operation example of the slave apparatus 10 according to the second embodiment of the present disclosure with reference to FIG. 10. FIG. 10 is a flowchart illustrating an operation example of the controller 120 of the slave apparatus 10 according to the second embodiment of the present disclosure.

First, the slave apparatus 10 comes into operation in accordance with an operation of a user on the master apparatus 30. The first acquisition unit 121 of the controller 120 then acquires a vibration signal measured by the sensor unit 110 (step S2000), and transmits the vibration signal to the output control unit 123-2. The A/D 124 of the output control unit 123-2 converts the vibration signal from an analog signal to a digital signal (step S2004), and transmits the converted vibration signal to the noise reduction section 125. The noise reduction section 125 removes, by filtering, noise from the vibration signal converted into a digital signal (step S2008).

In addition, in parallel with the above-described processes in steps S2000, 2004, and 2008, the second acquisition unit 122 of the controller 120 acquires the distance between the contact part 142 and the target measured by the sensor unit 110 (step S2012).

After the parallel processes are finished, the weight decision section 127-2 decides a weight corresponding to the distance acquired in step S2012 (step S2016). After the weight is decided, the output control unit 123-2 outputs a vibration signal multiplied by the weight by the weight decision section 127-2 and converted from a digital signal to an analog signal by the D/A 128 to the master apparatus 30 as an output signal (step S2020).

The operation example of the slave apparatus 10 according to the second embodiment of the present disclosure has been described above with reference to FIG. 10.

The second embodiment of the present disclosure has been described above with reference to FIGS. 7 to 10.

As described above, the information processing device according to the first embodiment is able to cut off an output signal in a case where the contact part 142 and a target are not in contact with each other. It is therefore sufficient if a user uses the information processing device according to the first embodiment in a case where the user wishes to acquire only a tactile sensation of contact. In addition, the information processing device according to the second embodiment is able to output an output signal even in a case where the contact part 142 and a target are not in contact with each other. It is therefore sufficient if a user uses the information processing device according to the second embodiment in a case where the user wishes to measure sound and vibration around a target.

Subsequently, a third embodiment of the present disclosure is described.

### 5. THIRD EMBODIMENT

In an information processing device according to the third embodiment, an output control unit 123-3 of the controller 120 makes a determination of contact as in the first embodiment, and further decides a weight in accordance with the distance between the contact part 142 and a target as in the second embodiment.

### <5.1. Configuration Example of Output Control Unit 123-3>

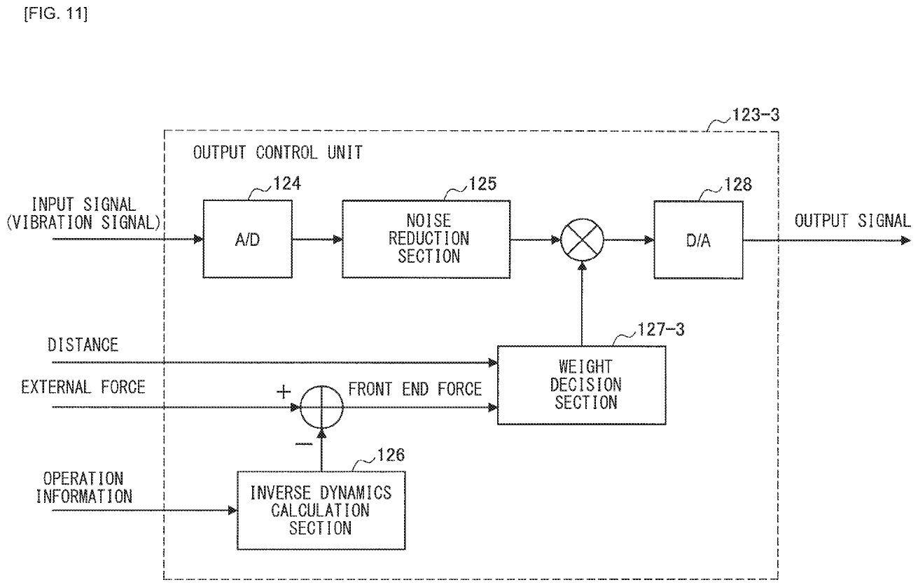

The following describes a configuration example of an output control unit according to the third embodiment of the present disclosure with reference to FIG. 11. FIG. 11 is an explanatory diagram illustrating the configuration example of the output control unit according to the third embodiment of the present disclosure. As illustrated in FIG. 11, the output control unit 123-3 includes the A/D 124, the noise reduction section 125, the inverse dynamics calculation section 126, a weight decision section 127-3, and the D/A 128.

### (1) A/D 124

The function of the A/D 124 is the same as the function described in <3.1. Configuration Example of Output Control Unit 123-1>, and the description thereof is omitted in this chapter.

### (2) Noise Reduction Section 125

The function of the noise reduction section 125 is the same as the function described in <3.1. Configuration Example of Output Control Unit 123-1>, and the description thereof is omitted in this chapter.

### (3) Inverse Dynamics Calculation Section 126

The function of the inverse dynamics calculation section 126 is the same as the function described in <3.1. Configuration Example of Output Control Unit 123-1>, and the description thereof is omitted in this chapter.

### (4) Weight Decision Section 127-3

The function of the weight decision section 127-3 has the function of deciding a weight in accordance with distance described in <4.1. Configuration Example of Output Control Unit 123-2> in addition to the function of deciding a weight in accordance with a determination of contact described in <3.1. Configuration Example of Output Control Unit 123-1>. It is to be noted that the respective functions are the same as the functions described in <3.1. Configuration Example of Output Control Unit 123-1> and <4.1. Configuration Example of Output Control Unit 123-2>, and the descriptions thereof are omitted in this chapter. The weight decision section 127-3 according to the third embodiment is, however, different from the weight decision sections 127-3 according to the first embodiment and the second embodiment in that the weight decision section 127-3 according to the third embodiment is able to use the above-described two functions in combination. In addition, the weight decision section 127-3 is also different in that the weight decision section 127-3 receives front end force and distance information as input information to use the above-described two functions in combination.

As described above, the weight decision section 127-3 according to the third embodiment is able to use the above-described two functions in combination, and it is thus possible to increase the weight deciding accuracy more than in the first embodiment and the second embodiment.

### (5) D/A 128

The function of the D/A 128 is the same as the function described in <3.1. Configuration Example of Output Control Unit 123-1>, and the description thereof is omitted in this chapter.

The configuration example of the output control unit 123-3 according to the third embodiment of the present disclosure has been described above with reference to FIG. 11. Subsequently, an operation example of the slave apparatus 10 according to the third embodiment of the present disclosure is described.

### <5.2. Operation Example of Slave Apparatus 10>

The following describes the operation example of the slave apparatus 10 according to the third embodiment of the present disclosure. The operation of the slave apparatus 10 according to the third embodiment is the combination of the operations of the slave apparatuses 10 according to the first embodiment and the second embodiment. For example, the second acquisition unit 122 also acquires the distance between a target and the contact part 142 in parallel with the processes in steps S1012 to S1020 illustrated in FIG. 6. Then, in a case where it is determined as a determination of contact that the target and the contact part 142 are not in contact with each other (step S1036), the weight decision section 127-3 does not decide a weight as 0 as in step S1040, but decides a weight corresponding to the distance as in step S2016 illustrated in FIG. 10.

The operation example of the slave apparatus 10 according to the third embodiment of the present disclosure has been described above.

The third embodiment of the present disclosure has been described above with reference to FIG. 11. Subsequently, modification examples of the embodiment of the present disclosure are described.

### 6. MODIFICATION EXAMPLES

The following describes the modification examples of the embodiment of the present disclosure. It is to be noted that, the respective modification examples described below may be separately applied to the embodiment of the present disclosure, or may be applied to the embodiment of the present disclosure in combination. In addition, the respective modifications may be applied instead of the configuration described in the embodiment of the present disclosure, or may be applied in addition to the configuration described in the embodiment of the present disclosure.

The method for a weight decision section 127 to make a determination of contact on the basis of information measured by the sensor unit 110 has been described in the above-described embodiments, but the weight decision section 127 may also make a determination of contact on the basis of information regarding a sensor included in advance in the slave apparatus 10.

For example, the weight decision section 127 may make a determination of contact on the basis of information acquired by a sensor included in advance in the slave apparatus 10. Specifically, for example, the weight decision section 127 may make a determination of contact on the basis of information of a camera image acquired by a camera included in advance in the slave apparatus 10. In addition, for example, the weight decision section 127 may make a determination of contact on the basis of a result of machine learning of information acquired by a sensor included in advance in the slave apparatus 10.

In addition, for example, the weight decision section 127 may make a determination of contact on the basis of control information for controlling a sensor included in advance in the slave apparatus 10. Specifically, for example, the weight decision section 127 may make a determination of contact on the basis of contact information such as disturbance/acceleration/jerk of a motor.

In addition, the weight decision section 127 may combine results of information processing on respective pieces of information acquired by a plurality of sensors included in advance in the slave apparatus 10, and make a determination of contact on the basis of the results.

As described above, the weight decision section 127 makes a determination of contact on the basis of information regarding a sensor included in advance in the slave apparatus 10, thereby allowing the slave apparatus 10 to achieve the above-described processes with no new sensor added.

The modification examples of the embodiment of the present disclosure have been described above. Subsequently, a hardware configuration according to an embodiment of the present disclosure is described.

### 7. HARDWARE CONFIGURATION

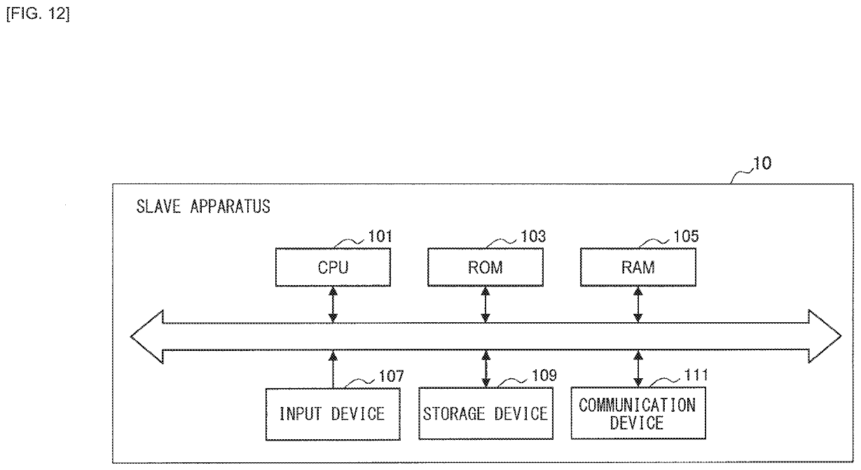

Finally, the hardware configuration of the slave apparatus 10 according to the present embodiment is described with reference to FIG. 12. FIG. 12 is a block diagram illustrating an example of the hardware configuration of the slave apparatus 10 according to the present embodiment. Information processing by the slave apparatus 10 according to the present embodiment is achieved in cooperation between software and hardware described below.

The slave apparatus 10 includes CPU (Central Processing Unit) 101, ROM (Read Only Memory) 103, and RAM (Random Access Memory) 105. In addition, the slave apparatus 10 includes an input device 107, a storage device 109, and a communication device 111.

The CPU 101 functions as an arithmetic processing device and a control device, and controls the overall operation in the slave apparatus 10 in accordance with various programs. In addition, the CPU 101 may be a microprocessor. The ROM 103 stores programs, arithmetic parameters, and the like used by the CPU 101. The RAM 105 temporarily stores programs used in execution of the CPU 101, parameters appropriately changed in the execution, and the like. These are coupled to each other by a host bus including a CPU bus or the like. The CPU 101, the ROM 103, and the RAM 105 may achieve, for example, the functions of the controller 120 described with reference to FIG. 2.

The input device 107 includes an input means such as a touch panel, a button, a camera, a microphone, a sensor, a switch, and a lever for a user to input information, an input control circuit that generates an input signal on the basis of the input from the user and outputs the input signal to the CPU 101, and the like. A user of the slave apparatus 10 operates, for example, the master apparatus 30 to bring the slave apparatus 10 into operation. This causes the input device 107 to acquire data and hereby inputs various kinds of data to the slave apparatus 10 or instructs the slave apparatus 10 about a processing operation. The input device 107 may achieve, for example, the function of the sensor unit 110 described with reference to FIG. 2.

The storage device 109 is a device for data storage. The storage device 109 may include a storage medium, a recording device that records data in the storage medium, a readout device that reads out data from the storage medium, a deletion device that deletes data recoded in the storage medium, and the like. The storage device 109 includes, for example, HDD (Hard Disk Drive) or SSD (Solid Storage Drive). Alternatively, the storage device 109 includes, a memory or the like having the equivalent function. This storage device 109 drives a storage, and stores a program executed by the CPU 101 and various kinds of data. The storage device 109 may achieve, for example, the function of the storage unit 130 described with reference to FIG. 2.

The communication device 111 is, for example, a communication interface including a communication device and the like for coupling the slave apparatus 10 and the master apparatus 30. Examples of the communication interface include a near field communication interface such as Bluetooth (registered trademark) or ZigBee (registered trademark), or a communication interface such as wireless LAN (Local Area Network), Wi-Fi (registered trademark), or a mobile communication network (LTE or 3G). In addition, the communication device 111 may be a wired communication device that performs wired communication.

The hardware configuration of the slave apparatus 10 has been described above with reference to FIG. 12.

### 8. CONCLUSION

As described above, the information processing device according to the present disclosure acquires a vibration signal measured by a vibration sensor from information measured by a sensor included in the slave apparatus 10. In addition, the information processing device acquires a measurement result regarding the distance between a target of sensing of the vibration sensor and the contact part 142 of the slave apparatus 10 that comes into contact with the target. The information processing device then decides a weight on the basis of the acquired measurement result, and applies the weight to the vibration signal, thereby making it possible to control the magnitude of an output signal outputted to the master apparatus 30.

As a result, vibration outputted from the master apparatus 30 and presented to the user changes in accordance with the distance between the contact part 142 of the slave apparatus 10 and the target, and it is possible to make the user feel less strange. It is desirable in particular to decide a weight applied to a vibration signal in accordance with a result of a determination of the contact between the contact part 142 of the slave apparatus 10 and the target. In that case, it is possible to cut off an output signal and refrain vibration from being presented to the user when the contact part 142 of the slave apparatus 10 and the target are not in contact with each other. As a result, it is possible to make the user feel further less strange. It is therefore possible to provide a novel and improved information processing device, information processing method, and program each of which makes it possible to make a user feel less strange.

A preferred embodiment(s) of the present disclosure has/have been described above in detail with reference to the accompanying drawings, but the technical scope of the present disclosure is not limited to such an embodiment(s). A person skilled in the art may find various alterations and modifications within the scope of the appended claims, and it should be understood that they will naturally come under the technical scope of the present disclosure.

In addition, the series of processes by each device described herein may be achieved by using any of software, hardware, and the combination of software and hardware. A program included in the software is stored in advance, for example, in a recording medium (non-transitory medium: non-transitory media) provided inside or outside each device. Then, each program is read by RAM, for example, when executed by a computer, and is executed by a processor such as CPU.

In addition, the processes described by using the flowcharts and the sequence diagrams in this specification do not necessarily have to be executed in the illustrated order. Some of the processing steps may be executed in parallel. In addition, an additional processing step may be adopted, and some of the processing steps may be omitted.

In addition, the effects described herein are merely illustrative and exemplary, and not limitative. That is, the technology according to the present disclosure may exert other effects that are apparent to those skilled in the art from the description herein, in addition to the above-described effects or in place of the above-described effects.

It is to be noted that the following configurations also fall within the technical scope of the present disclosure.

(1)

An information processing device including:

a first acquisition unit that acquires a vibration signal measured by a vibration sensor included in a slave apparatus;

a second acquisition unit that acquires a measurement result regarding distance between a target of sensing of the vibration sensor and a contact part of the slave apparatus, the contact part coming into contact with the target; and

a controller that outputs an output signal to a master apparatus, the output signal being obtained by applying a weight to the vibration signal, the weight corresponding to the measurement result.

(2)

The information processing device according to (1), in which the controller makes a determination of contact between the contact part and the target, and decides the weight on the basis of a result of the determination.

(3)

The information processing device according to (2), in which, in a case where the controller determines that the contact part and the target are in contact with each other, the controller outputs the output signal.

(4)

The information processing device according to any one of (2) to (3), in which, in a case where the controller determines that the contact part and the target are not in contact with each other, the controller cuts off the output signal.

(5)

The information processing device according to any one of (1) to (4), in which the controller continuously changes the weight in accordance with the distance between the contact part and the target.

(6)

The information processing device according to (1), in which the controller removes, by using a filter, a frequency component other than a frequency component corresponding to a tactile sensation of a human or a predetermined frequency component stored in advance from the vibration signal.

(7)

The information processing device according to (1), in which

the slave apparatus further includes a biological sensor that measures biological information, and

the controller makes a determination of contact between the contact part and the target on the basis of the biological information.

(8)

The information processing device according to (2), in which

the slave apparatus further includes a force sensor that measures force applied to the contact part, and

the second acquisition unit acquires the force as the measurement result, the force being measured by the force sensor.

(9)

The information processing device according to (8), in which the controller corrects the force with inverse dynamics calculation, and then makes the determination of the contact, the force being measured by the force sensor.

(10)

The information processing device according to (1), in which the second acquisition unit acquires the distance between the target and the contact part.

(11)

An information processing method that is executed by a processor, the information processing method including:

acquiring a vibration signal measured by a vibration sensor included in a slave apparatus;

acquiring a measurement result regarding distance between a target of sensing of the vibration sensor and a contact part of the slave apparatus, the contact part coming into contact with the target; and

outputting an output signal to a master apparatus, the output signal being obtained by applying a weight to the vibration signal, the weight corresponding to the measurement result.

(12)

A program for causing a computer to function as

a first acquisition unit that acquires a vibration signal measured by a vibration sensor included in a slave apparatus,

a second acquisition unit that acquires a measurement result regarding distance between a target of sensing of the vibration sensor and a contact part of the slave apparatus, the contact part coming into contact with the target, and

a controller that outputs an output signal to a master apparatus, the output signal being obtained by applying a weight to the vibration signal, the weight corresponding to the measurement result.

### REFERENCE SIGNS LIST

10 slave apparatus
30 master apparatus
110 sensor unit
120 controller
121 first acquisition unit
122 second acquisition unit
123 output control unit
124 A/D
125 noise reduction section
126 inverse dynamics calculation section
127 weight decision section
128 D/A
130 storage unit
140 front end part
142 contact part
310 active joint part
320 passive joint part
330 operation body
340 force sensor

1. An information processing device comprising:
a first acquisition unit that acquires a vibration signal measured by a vibration sensor included in a slave apparatus;
a second acquisition unit that acquires a measurement result regarding distance between a target of sensing of the vibration sensor and a contact part of the slave apparatus, the contact part coming into contact with the target; and
a controller that outputs an output signal to a master apparatus, the output signal being obtained by applying a weight to the vibration signal, the weight corresponding to the measurement result.

2. The information processing device according to claim 1, wherein the controller makes a determination of contact between the contact part and the target, and decides the weight on a basis of a result of the determination.

3. The information processing device according to claim 2, wherein, in a case where the controller determines that the contact part and the target are in contact with each other, the controller outputs the output signal.

4. The information processing device according to claim 2, wherein, in a case where the controller determines that the contact part and the target are not in contact with each other, the controller cuts off the output signal.

5. The information processing device according to claim 1, wherein the controller continuously changes the weight in accordance with the distance between the contact part and the target.

6. The information processing device according to claim 1, wherein the controller removes, by using a filter, a frequency component other than a frequency component corresponding to a tactile sensation of a human or a predetermined frequency component stored in advance from the vibration signal.

7. The information processing device according to claim 1, wherein
the slave apparatus further includes a biological sensor that measures biological information, and
the controller makes a determination of contact between the contact part and the target on a basis of the biological information.

8. The information processing device according to claim 2, wherein
the slave apparatus further includes a force sensor that measures force applied to the contact part, and
the second acquisition unit acquires the force as the measurement result, the force being measured by the force sensor.

9. The information processing device according to claim 8, wherein the controller corrects the force with inverse dynamics calculation, and then makes the determination of the contact, the force being measured by the force sensor.

10. The information processing device according to claim 1, wherein the second acquisition unit acquires the distance between the target and the contact part.

11. An information processing method that is executed by a processor, the information processing method comprising:
acquiring a vibration signal measured by a vibration sensor included in a slave apparatus;
acquiring a measurement result regarding distance between a target of sensing of the vibration sensor and a contact part of the slave apparatus, the contact part coming into contact with the target; and
outputting an output signal to a master apparatus, the output signal being obtained by applying a weight to the vibration signal, the weight corresponding to the measurement result.

12. A program for causing a computer to function as
a first acquisition unit that acquires a vibration signal measured by a vibration sensor included in a slave apparatus,
a second acquisition unit that acquires a measurement result regarding distance between a target of sensing of the vibration sensor and a contact part of the slave apparatus, the contact part coming into contact with the target, and
a controller that outputs an output signal to a master apparatus, the output signal being obtained by applying a weight to the vibration signal, the weight corresponding to the measurement result.
